(19) United States
(12) Patent Application Publication
(10) Pub. No.: US 2010/0003264 A1
(43) Pub. Date: Jan. 7, 2010

(54) Reduction of Ophthalmalogic Neovascularization

(75) Inventors: Rajendra S. Apte, Clayton, MO (US); Thomas A. Ferguson, Kirkwood, MO (US)

Correspondence Address: SENNIGER POWERS LLP (WSHU), 100 NORTH BROADWAY, 17TH FLOOR, SAINT LOUIS, MO, 63102, US

(73) Assignee: WASHINGTON UNIVERSITY, St. Louis, MO (US)

(21) Appl. No.: 11/909,405

(22) PCT Filed: Mar. 24, 2006
(86) PCT No.: PCT/US2006/011075

§ 371 Date: Sep. 24, 2009

Related U.S. Application Data
(60) Provisional application No. 60/664,852, filed on Mar. 24, 2005.

(51) Int. Cl. A61K 39/395; A61K 48/00; A61K 31/352; A61P 27/02; A61K 35/12

(52) U.S. Cl. 424/158.1; 514/44 A; 514/456; 424/93.7

(57) ABSTRACT

The present invention generally relates to methods for treatment of neovascularization in various tissues of a patient's eye. One aspect of the invention is a method of treating a patient for ophthalmologic neovascularization by administering an anti-interleukin-10 agent to the eye of a patient in need thereof to decrease the amount of interleukin-10 in the eye. Another aspect of the invention is a method of treating a patient for ophthalmologic neovascularization by administering isolated macrophages to the eye of a patient in need thereof to decrease a volume of a neovascularization complex within the treated eye.

### FIELD OF THE INVENTION

The present invention generally relates to methods for treatment of neovascularization in various tissues of a patient's eye.

### BACKGROUND

Angiogenesis is the formation of new capillary blood vessels leading to neovascularization. Though angiogenesis is a normal process for the development or maintenance of the vasculature, pathological conditions (i.e., angiogenesis dependent diseases) arise where blood vessel growth is actually harmful. In the eye, the neovascularization (de novo proliferation of endothelium and blood vessels) of ocular structures during disease or injury can disrupt ocular physiological balance and can lead to vision loss and/or blindness.

Any abnormal growth of blood vessels in the eye can scatter and block the incident light prior to reaching the retina. Neovascularization can occur at almost any site in the eye and significantly alter ocular tissue function. Antiangiogenic therapy would allow modulation in such angiogenesis-associated diseases having excessive vascularization. Some of the most threatening ocular neovascular diseases are those which involve the retina. For example, many diabetic patients develop a retinopathy which is characterized by the formation of leaky, new blood vessels on the anterior surface of the retina and in the vitreous causing proliferative vitreoretinopathy. A subset of patients with age related macular degeneration develops choroidal neovascularization which leads to their eventual blindness. Choroidal neovascularization is the development of new blood vessels originating in the choroid and encroaching on to the sub-retinal space.

Although thermal laser photocoagulation, ocular photodynamic therapy with verteporfin, and intravitreal anti-VEGF therapy with pegaptanib Na have offered some treatment options in the management of certain subsets of choroidal neovascularization, these treatments are at best palliative and designed to limit further visual loss but not to reverse retinochoroidal damage (Ambati et al. (2003) Surv Ophthalmol 48: 257). These therapies are only partially effective and generally only slow neovascularization and the progress of the overall disease. In addition, they can cause severe side effects if used over a relatively long period of time.

Glucocorticoids have also been shown to inhibit angiogenesis. However, the use of glucocorticoid therapy in general is complicated by the inherent problems associated with steroid applications such as elevated intraocular pressure. Still other therapies have included the use of protamine (Taylor (1982) Nature 297: 307-312), the use of calcitriol (European Journal of Pharmacology (1990) 178: 247-250), and the use of the antibiotic, fumagillin and its analogs, disclosed in EP 354787.

Other attempts have been made to provide therapies for the prevention or treatment of pathological angiogenesis. For example, angiostatic steroids functioning to inhibit angiogenesis in the presence of heparin or specific heparin fragments have been described (Crum et al. (1985) Science 230: 1375-1378; Kitazawa (1976) American Journal of Ophthalmology 82: 492-493). Another group of angiostatic steroids useful in inhibiting angiogenesis is disclosed in Clark et al., U.S. Pat. No. 5,371,078.

Interleukin-10 (IL-10) is a cytokine produced by activated macrophages and some helper T cells. The major function of IL-10 is to inhibit activated macrophages and therefore maintain homeostatic control of innate and cell-mediated immune reactions (Moore et al. (2001) Annu. Rev. Immunol. 19: 683-765). By inhibiting proliferation and effector functions of activated macrophages, IL-10 serves to control cell-mediated immune responses through feedback function. Small interference RNA has been shown to be capable of modulating IL-10 gene expression in dendritic cells (Liu et al. (2004) Eur. J. Immunol. 2004. 34: 1680-1687). Similarly, antisense oligonucleotides specific for interleukin-10 mRNA have also been shown to be capable of modulating IL-10 in the treatment of chronic lymphocytic leukemia (Raveche, U.S. Pat. No. 6,184,372; Peng et al. (1995) Leu. Res. 19: 159-167).

Interleukin-10 Receptor (IL-10-R) is a Type II cytokine receptor, classified as such based upon conserved extracellular domain structure. IL-10-R contains two extracellular domains consisting of one ligand-binding polypeptide chain and one signal-transducing chain. The effects of IL-10 are transduced through binding to IL-10-R.

The host immune effector armamentarium is comprised of innate and adaptive immune systems. Macrophages are key players in the innate immune system that among others have been shown to play a crucial role in the development of choroidal neovascularization (Sakurai et al. (2003) Invest Ophthalmol Vis Sci 44: 3578; Espinosa-Heidmann et al. (2003) Invest Ophthalmol Vis Sci 44: 3586; Ambati et al. (2003) Nature Medicine 9: 1390-1397).

Agents which inhibit neovascularization are known by a variety of terms such as angiostatic, angiolytic, angiogenesis inhibitors or angiotropic agents.

### SUMMARY OF THE INVENTION

One aspect of the invention provides a method for the treatment of abnormal angiogenesis within tissues of the eye through administration of anti-interleukin-10 agents and/or anti-interleukin-10-receptor agents. Such treatment can be prophylactic or therapeutic and directed towards ophthalmologic neovascularization, for example choroidal neovascularization. The method of treatment involves administering an anti-interleukin-10 agent and/or anti-interleukin-10-receptor agent to a patient's eye. Generally, the patient will be in need of such therapy as diagnosed during, for example, a routine eye exam. In the course of therapy, the anti-interleukin-10 agent decreases the levels of interleukin-10 in the tissues of the eye. Similarly, the anti-interleukin-10-receptor agent decreases the levels of active interleukin-10-receptors in the tissue of the eye. Lower levels of interleukin-10 (or receptor) results in a decreased volume of neovascularization complex within the treated eye.

Another aspect of the invention provides a method for the treatment of abnormal angiogenesis within tissues of the eye through administration of isolated macrophages. Such treatment can be prophylactic or therapeutic and directed towards ophthalmologic neovascularization, for example choroidal neovascularization. The method of treatment involves administering isolated macrophages to a patient's eye. Generally, the patient will be in need of such therapy as diagnosed during, for example, a routine eye exam. In the course of therapy, the isolated macrophages effect a decreased volume of neovascularization complex within the treated eye.

Other objects and features will be in part apparent and in part pointed out hereinafter.

### BRIEF DESCRIPTION OF THE DRAWINGS

FIG. 1 is a bar graph depicting volume of the neovascular complex in a murine model of laser-induced choroidal neovascularization in wild type mice (IL-10+/+) and IL-10 knockout mice (IL-10−/−).

FIG. 2 is a bar graph depicting volume of the neovascular complex in a murine model of laser-induced choroidal neovascularization in wild type mice and wild type mice treated with neutralizing IL-10 antibodies.

FIG. 3 is a bar graph depicting volume of the neovascular complex in a murine model of laser-induced choroidal neovascularization in wild type mice with wild type bone marrow (B6/B6); wild type mice with IL-10 knock out bone marrow (B6/IL-10−/−); IL-10 knock out mice with wild type bone marrow (IL-10−/−/B6); and IL-10 knock out mice with IL-10 knock out bone marrow (IL-10−/−/IL-10−/−).

FIG. 4 is a bar graph depicting volume of the neovascular complex in a murine model of laser-induced choroidal neovascularization in IL-10 knock out mice and IL-10 knock out mice treated with exogenous IL-10 at day zero and day three after laser treatment.

FIG. 5 is a bar graph depicting the number of cells specific for the CD11b+ macrophage marker in sclerochoroidal flat mounts prepared seven days after laser treatment from wild type or IL-10 knock out mouse eyes.

FIG. 6 is a bar graph depicting the number of cells specific for the CD11b+ macrophage marker in sclerochoroidal flat mounts prepared seven days after laser treatment from wild type mouse eyes or wild type mouse eyes treated with neutralizing IL-10 antibody.

FIG. 7 is a bar graph depicting volume of the neovascular complex in a murine model of laser-induced choroidal neovascularization in wild type mouse eyes injected with PBS control or macrophages (CD11b+ marker) at 1×10⁵ or 5×10⁵ macrophages per injection on the same day as the laser treatment.

FIG. 8 is a bar graph depicting volume of the neovascular complex in a murine model of laser-induced choroidal neovascularization in wild type mouse eyes injected with PBS control, T-cells (CD3+ marker), macrophages (CD11b+ marker), or dendritic cells (CD11c+ marker) on the same day as the laser treatment.

FIG. 9 is a bar graph depicting the volume of the neovascular complex in a murine model of laser-induced choroidal neovascularization in wild type B6 mice injected with CD11b+ macrophages from either B6 wild type mice, FAS-deficient mice (B6-lpr), or FasLigand-deficient mice (B6-gld).

FIG. 10 is a bar graph depicting the number of cells specific for the CD11b+ macrophage marker in sclerochoroidal flat mounts. Macrophages from B6-wt or B6-gld mice were labeled with CFSE and injected into the vitreous cavity on the day of laser treatment. The number of macrophages per laser lesion were counted on day 3. (p=0.24).

FIG. 11 is a bar graph depicting the volume of the neovascular complex in a murine model of laser-induced choroidal neovascularization in IL-10−/− mice injected with anti-CD11b, anti-F4/80, or control IgG On days −1, 0, and +1. Choroidal neovascularization volumes were determined on day 7.

FIG. 12 is several cross section images of retina of transgenic (Tg) mice overexpressing IL-10 in the retinal pigment epithelium. Cross sections of control littermate (FIG. 12A) or VMD2-IL-10 Tg mice (FIG. 12B) were stained with anti-IL-10 and examined by confocal microscopy. FIG. 12C is an H & E stain of VMD2-IL-10 Tg mice (magnification=200×).

FIG. 13 is a bar graph depicting the volume of the neovascular complex in transgeneic mice overexpressing IL-10 in the retinal pigment epithelium. VMD2-IL-10 Tg mice or or littermate controls were subjected to laser treatment and CNV volumes were determined on day 7

FIG. 14 is a bar graph depicting the percent cell death of purified CD11b+ cells. The cells were cultured overnight with LPS or necrotic retina and tested for killing against L1210-Fas.

FIG. 15 is a graph depicting the expression of CD95L as determined by flow cytometry. The cells were cultured overnight with LPS (represented by the dotted line) or necrotic retina (represented by the solid line). Untreated cells are represented by the shaded area.

FIG. 16 is a bar graph depicting the volume of the neovascular complex in a murine model of laser-induced choroidal neovascularization. Bone marrow chimeras were generated to test the source IL-10. Bone marrow (BM) was from B6 or IL-10−/− mice. Recipient mice were either B6 or IL-10−/− mice. IL-10−/− mice that were reconstituted with B6 bone marrow showed control levels of choroidal neovascularization. In contrast, B6 mice that were reconstituted with IL-10−/− bone marrow showed levels of choroidal neovascularization similar to IL-10−/− mice.

FIG. 17 is several cross section images of retina of IL-10−/− mice and wild type B6 mice seven days following laser treatment. Representative lesions from a B6 eye (FIG. 17A) and an IL-10−/− eye (FIG. 17B) depict the differences in choroidal neovascularization.

FIG. 18 is several confocal microscopy images of neovascular complexes in various mice. On day 7 following laser treatment, whole mount stains were performed to determine the cells present in the area of the neovascular complex. Stains for CD11b were performed on B6 mice (FIG. 18A), IL-10−/− mice (FIG. 18B), and B6 mice treated with neutralizing anti-IL-10 (FIG. 18C) using PE conjugated antibody. Images were taken by confocal microscopy (magnification=200×) centered on the laser lesion. Dual stains were performed on day 7 following laser treatment using FITC conjugated anti-CD11b and PE-conjugated anti-F4/80 (magnification=400×), confirming that the infiltrating cells were macrophages (FIG. 18D).

FIG. 19 is a confocal microscopy image of a representative lesion from a B6 wild-type mouse that was intravenously injected with GFP-labeled liposomes on the day of laser treatment. Presence of liposomes in the developing blood vessels within the laser lesions was analyzed on day 3.

### DETAILED DESCRIPTION OF THE INVENTION

In accordance with the inventors' finding that loss of anti-inflammatory cytokine IL-10 from hematopoetic cells results in significant reduction in neovascularization, several aspects of the invention are methods for the treatment, prophylactic or therapeutic, of neovascularization, for example choroidal neovascularization, through targeting IL-10 and IL-10 receptors. The method can be used, for example as a prophylactic to protect, in whole or in part, against neovascularization. The method can also be used, for example, therapeutically to ameliorate neovascularization and to protect, in whole or in part, against further neovascularization.

A determination of the need for treatment will typically be assessed during an eye exam. Such exams routinely include, for example, a medical history, vision testing, external examination, pupillary examination, intraocular pressure testing, a slit lamp examination of the anterior segment of the eye, and a pharmacologic dilation of the pupils in order to perform a biomicroscopic examination of the posterior segment of the eye. The posterior segment examination may reveal, for example, whether the patient has a disease or indication of a disease related to neovascularization that is amenable to therapeutic treatment described herein. Regardless of the means by which the need is identified, patients with an identified need of therapy with anti-IL10 agents, anti-IL-10-R agents, or macrophage injection will generally fall into one of several classes: (i) patients with a diagnosed disease or indication of disease amenable to therapeutic treatment described herein; (ii) patients who have been treated, are being treated, or will be treated for neovascularization with laser photocoagulation or photodynamic therapy; or (iii) patients with persistent or reoccurring neovascularization after surgical removal of an existing neovascularization.

Disease states indicative of a need for therapy with anti-IL10 agents, anti-IL-10-R agents, and/or macrophage injection and disease states amenable to treatment with anti-IL10 agents, anti-IL-10-R agents, and/or macrophage injection include, for example, intraocular melanoma; age-related macular degeneration; diabetic retinopathy; and retinopathy of prematurity in infants. Other examples of such disease states include: choroidal neovascularization due to histoplasmosis and pathological myopia; choroidal neovascularization that results from angioid streaks; anterior ischemic optic neuropathy; bacterial endocarditis; Best's disease; birdshot retinochoroidopathy; choroidal hemangioma; choroidal nevi; choroidal nonperfusion; choroidal osteomas; choroidal rupture; choroideremia; chronic retinal detachment; coloboma of the retina; Drusen; endogenous Candida endophthalmitis; extrapapillary hamartomas of the retinal pigmented epithelium; fundus flavimaculatus; idiopathic, macular hole, malignant melanoma; membranproliferative glomerulonephritis (type II); metallic intraocular foreign body; morning glory disc syndrome; multiple evanescent white-dot syndrome (MEWDS); neovascularization at ora serrata; operating microscope burn; optic nerve head pits; photocoagulation; punctate inner choroidopathy; radiation retinopathy; retinal cryoinjury; retinitis pigmentosa; retinochoroidal coloboma; rubella; sarcoidosis; serpiginous or geographic choroiditis; subretinal fluid drainage; tilted disc syndrome; Taxoplasma retinochoroiditis; tuberculosis; or Vogt-Koyanagi-Harada syndrome, among others.

In one aspect of the invention, the method comprises reducing the level of IL-10 in ophthalmologic tissue by, for example, administering an anti-IL-10 agent in an amount sufficient to treat neovascularization in tissues of the eye. The treatment can be prophylactic or therapeutic. In one study, knock-out mice incapable of producing IL-10 were unable to produce abnormal blood vessels in the eye after a krypton laser was used in a manner that would normally induce choroidal neovascularization (see e.g. Example 1). Exogenous IL-10 administered intravitreously on the day of laser or three days after laser reversed the inhibition choroidal neovascularization (see e.g., Example 4). These results demonstrate the efficacy of inactivating IL-10 in the eye to decrease the occurrence of choroidal neovascularization. Further experiments showed that wild type mice treated with neutralizing IL-10 antibody to deplete levels of active IL-10 showed the same inability to produce abnormal blood vessels in the eye after laser induced choroidal neovascularization (see e.g. Example 2). These results demonstrate that both knocking out levels of IL-10 as well as knocking down levels of IL-10 in the eye inhibits choroidal neovascularization.

Bone marrow chimeras were generated to test the contribution of hematopoetic cells. Inhibition of choroidal neovascularization in IL-10 knock-out mice was reversed after bone marrow chimera experiments in which the knock-out mice bone marrow was replaced with wild type (i.e., IL-10 producing) bone marrow (see e.g. Example 3). Furthermore, bone marrow not having the capacity of IL-10 formation successfully inhibited the ability of wild type mice to form choroidal neovascularization after generation of chimeras (see e.g. Example 3). From these experiments it was demonstrated that IL-10 supplied by bone marrow derived cells influence choroidal neovascularization.

When used in the treatments described herein, a therapeutically effective amount of one of the compounds of the present invention may be employed in pure form or, where such forms exist, in pharmaceutically acceptable salt form and with or without a pharmaceutically acceptable excipient. For example, the compound of the invention can be administered in a sufficient amount to inhibit formation of new blood vessels or reduce the number of blood vessels which are involved in the pathological condition at a reasonable benefit/risk ratio applicable to any medical treatment. It will be understood, however, that the total daily usage of the compounds and compositions of the present invention will be decided by the attending physician within the scope of sound medical judgment.

The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose.

Administration of the anti-IL-10 agent can occur as a single event or over a time course of treatment. For example, anti-IL-10 agent can be injected daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

According to the methods presented herein, prophylactic and therapeutic treatment of ophthalmologic neovascularization can be effected through reduction or down-regulation of IL-10 through administration of anti-IL-10 agents. Such anti-IL-10 agents include, for example, an IL-10 antisense nucleic acid molecule, an IL-10 small interfering RNA, a neutralizing IL-10 antibody, an IL-10 inhibitor, or an IL-10 agonist. Similarly, prophylactic and therapeutic treatment of ophthalmologic neovascularization can be effected through reduction or down-regulation of IL-10-R through administration of anti-IL-10-R agents.

Antisense IL-10

The levels of interleukin-10 can be down-regulated by administering to the patient a therapeutically effective amount of an antisense oligonucleotide specific for interleukin-10 mRNA. The antisense oligonucleotide specific for interleukin-10 mRNA may span the region adjacent to the initiation site of interleukin-10 translation, preferably region 1-500, more preferably region 310-347, and most preferably region 315-342. In one example, the antisense oligonucleotide specific for interleukin-10 mRNA is any one of the antisense sequences described by U.S. Pat. No. 6,184,372.

An effective amount of the antisense oligonucleotide specific for interleukin-10 mRNA as isolated in a purified form may be generally that amount capable of inhibiting the production of IL-10 or reducing the amount produced or the rate of production of IL-10 such that a reduction in neovascularization occurs. IL-10 antisense oligonucleotides can be administered via intravitreous injection at a concentration of about 10 μg/day to about 3 mg/day. For example, administered dosage can be about 30 μg/day to about 300 μg/day. As another example, IL-10 antisense oligonucleotide can be administered at about 100 μg/day. Administration of IL-10 antisense oligonucleotides can occur as a single event or over a time course of treatment. For example, IL-10 antisense oligonucleotides can be injected daily, weekly, bi-weekly, or monthly. Time course of treatment can be from about a week to about a year or more. In one example, IL-10 antisense oligonucleotides are injected daily for one month. In another example, IL-10 antisense oligonucleotides are injected weekly for about 10 weeks. In a further example, IL-10 antisense oligonucleotides are injected every 6 weeks for 48 weeks.

RNA Interference

The levels of interleukin-10 can be down-regulated by RNA interference by administering to the patient a therapeutically effective amount of small interfering RNAs (siRNA) specific for IL-10. siRNA specific for IL-10 is commercially available from sources such as Ambion (Austin, Tex.). The siRNA can be administered to the subject by any means suitable for delivering the siRNA to the cells of the tissue at or near the area of neovascularization. For example, the siRNA can be administered by gene gun, electroporation, or by other suitable parenteral or enteral administration routes, such as intravitreous injection.

RNA interference is the process by which double stranded RNA (dsRNA) specifically suppresses the expression of a gene bearing its complementary sequence. Suppression of the IL-10 gene inhibits the production of the IL-10 protein. Upon introduction, the long dsRNAs enter a cellular pathway that is commonly referred to as the RNA interference (RNAi) pathway. First, the dsRNAs get processed into 20-25 nucleotide (nt) small interfering RNAs (siRNAs) by an RNase III-like enzyme called Dicer (initiation step). Then, the siRNAs assemble into endoribonuclease-containing complexes known as RNA-induced silencing complexes (RISCs), unwinding in the process. The siRNA strands subsequently guide the RISCs to complementary RNA molecules, where they cleave and destroy the cognate RNA (effecter step). Cleavage of cognate RNA takes place near the middle of the region bound by the siRNA strand. Preferably, the siRNA comprises short double-stranded RNA from about 17 nucleotides to about 29 nucleotides in length, preferably from about 19 to about 25 nucleotides in length, that are targeted to the target mRNA.

As an example, an effective amount of the siRNA can be an amount sufficient to cause RNAi-mediated degradation of the target mRNA, or an amount sufficient to inhibit the progression of angiogenesis in a subject. One skilled in the art can readily determine an effective amount of the siRNA of the invention to be administered to a given subject by taking into account factors such as the size and weight of the subject; the extent of the neovascularization or disease penetration; the age, health and sex of the subject; the route of administration; and whether the administration is regional or systemic. Generally, an effective amount of siRNA comprises an intercellular concentration at or near the neovascularization site of from about 1 nanomolar (nM) to about 100 nM, preferably from about 2 nM to about 50 nM, more preferably from about 2.5 nM to about 10 nM. It is contemplated that greater or lesser amounts of siRNA can be administered.

The siRNA can be targeted to any stretch of approximately 19-25 contiguous nucleotides in any of the IL-10 or IL-10-R mRNA target sequences. Target sequences can be selected from, for example, the sequence of human IL-10, Genebank access number: AY029171. Searches of the human genome database (BLAST) can be carried out to ensure that selected siRNA sequence will not target other gene transcripts. Techniques for selecting target sequences for siRNA are given, for example, in Elbashir et al. ((2001) Nature 411L 494-498). Thus, the sense strand of the present siRNA comprises a nucleotide sequence identical to any contiguous stretch of about 19 to about 25 nucleotides in the target mRNA of IL-10 or IL-10 receptor. Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon.

IL-10 Inhibitor

Production of IL-10 can be inhibited through the administration of an IL-10 inhibitor. One such inhibitor is a polymethoxylated flavone. Polymethoxylated flavones include those compounds described in U.S. Pat. No. 6,184,246. The polymethoxylated flavone can be administered by, for example, intravitreous injection. However, it should be understood that the amount of the polymethoxylated flavone actually administered ought to be determined in light of various relevant factors including the condition to be treated, the chosen route of administration, the age and weight of the individual patient, and the severity of the patient's symptom. Preferably, the dose is an IL-10 inhibiting amount (e.g., a quantity of polymethoxylated flavones capable of inhibiting the production of IL-10 or reducing the amount produced or the rate of production of IL-10). Methods of determining the effective concentrations are well known in the art. Generally, the injectable dose of IL-10 inhibitor can be between about 1.0 μg to about 1 g. For example, the IL-10 inhibitor dose can be between about 1 mg to about 100 mg. When the compositions are dosed topically, they will generally be in a concentration range of about 0.001 wt. % to about 5 wt. %, with 1-2 drops administered 1-5 times per day.

IL-10 Agonists

IL-10 agonists can be molecules which mimic IL-10 interaction with its receptors. Such may be analogs or fragments of IL-10, or antibodies against ligand binding site epitopes of the IL-10 receptors, or anti-idiotypic antibodies against particular antibodies which bind to receptor-interacting portions of IL-10.

Antagonists may take the form of proteins which compete for receptor binding, e.g., which lack the ability to activate the receptor while blocking IL-10 binding, or IL-10 binding molecules, e.g., antibodies. Anti-IL-10-R antibodies are commercially available from numerous sources including Fitzgerald Industries International (Concord, Mass.), Sigma-Aldrich (St. Louis, Mo.), and United States Biological (Swampscott, Mass.).

Neutralizing IL-10 Antibodies

Levels of IL-10 can be significantly reduced via administration of neutralizing IL-10 antibodies. Administration of anti-IL-10 antibodies have been shown to be therapeutically effective at treating choroidal neovascularization (see e.g., Example 2). Anti-IL-10 antibodies are commercially available from numerous sources including PeproTech (Rocky Hill, N.J.), GenWay Biotech, Inc (San Diego, Calif.), and Affinity BioReagents (Golden, Colo.). Antibodies can be raised to the IL-10 cytokine, fragments, and analogs, both in their naturally occurring forms and in their recombinant forms. Additionally, antibodies can be raised to IL-10 in either its active forms or in its inactive forms, the difference being that antibodies to the active cytokine are more likely to recognize epitopes which are only present in the active conformation. Anti-idiotypic antibodies are also contemplated in these methods, and could be potential IL-10 agonists.

Neutralizing IL-10 antibodies can be administered, for example, through intravitreous injection. The IL-10 antibodies can be injected at a concentration of from about 0.01 mg to about 5.0 mg per injection. For example, IL-10 antibodies can be injected at a concentration of about 0.05 mg to about 2.5 mg per injection. As another example, IL-10 antibodies can be injected at a concentration of about 0.1 mg to about 1 mg per injection. Preferably, IL-10 antibodies are injected at a concentration of about 0.3 mg to about 0.5 mg per injection. Administration of IL-10 antibodies can occur as a single event or over a time course of treatment. For example, IL-10 antibodies can be injected daily, weekly, bi-weekly, or monthly. Time course of treatment can be from about a week to about a year or more. In one example, IL-10 antibodies are injected every 6 weeks for a period of 48 weeks.

Antibodies, including binding fragments and single chain versions, against predetermined fragments of the desired antigens, e.g., cytokine, can be raised by immunization of animals with conjugates of the fragments with immunogenic proteins. Monoclonal antibodies are prepared from cells secreting the desired antibody. These antibodies can be screened for binding to normal or inactive analogs, or screened for agonistic or antagonistic activity. These monoclonal antibodies will usually bind with at least a K_{D} of about 1 mM, more usually at least about 300 μM, typically at least about 10 μM, more typically at least about 30 μM, preferably at least about 10 μM, and more preferably at least about 3 μM or better. Although the foregoing addresses IL-10, similar antibodies may be raised against other analogs, its receptors, and antagonists.

The antibodies, including antigen binding fragments, of this invention can have significant diagnostic or therapeutic value. They can be potent antagonists that bind to the IL-10 receptors and inhibit ligand binding to the receptor or inhibit the ability of IL-10 to elicit a biological response. IL-10 or fragments may be joined to other materials, particularly polypeptides, as fused or covalently joined polypeptides to be used as immunogens.

Isolated Macrophages

In another aspect of the invention, the method comprises injection of macrophages into ophthalmologic tissue in an amount sufficient to treat neovascularization in tissues of the eye prophylactically or therapeutically. The presence of macrophages has been demonstrated to be important for the inhibition of formation of abnormal blood vessels in adults. Macrophage migration is primarily from the blood stream. Thus there is limited numbers of passively distributed macrophage in any particular tissue. So, any localized collection of macrophages is usually due to active migration. Generally, macrophages move in response to an attractant produced in response to an injury or the like. IL-10 was shown to inhibit migration of macrophages into the neovascular complex (see e.g. Example 5-6). Wild type mice treated with anti-IL-10 antibody (i.e., decreased levels of IL-10) had significantly increased levels of macrophage as shown by staining of CD11b+ (a macrophage specific receptor). Thus, macrophages can be used proactively for the treatment or prophylxaxis of choroidal neovascularization. Macrophages can be subject to a positive selection protocol, such as magnetic tag antibody (SpinSep™, Stem Cell Technologies, Inc.). The isolated macrophages can then be injected into the eye, thereby inhibiting formation of blood vessels (see e.g. Example 6).

The amount of isolated macrophages that can be used as an anti-angiogenic agent for the treatment of ocular neovascularization and related diseases includes an amount effective to inhibit the progression of angiogenesis in a subject. For example, the isolated macrophages can be intravitreously injected at a concentration of from about 1×10⁴ to about 1×10⁶ macrophages per injection. As another example, isolated macrophages can be intravitreously injected at a concentration of about 1×10⁵ to about 5×10⁵ macrophages per injection.

Therapeutic Administration

The anti-IL-10 agents or anti-IL-10-R agents can be used therapeutically either as exogenous materials or as endogenous materials. Exogenous agents are those produced or manufactured outside of the body and administered to the body. Endogenous agents are those produced or manufactured inside the body by some type of device (biologic or other) for delivery to within or to other organs in the body.

Exogenous Therapy

A safe and effective amount of anti-IL-10 agent, anti-IL-10-R agent, or isolated macrophages is, for example, that amount that would cause the desired therapeutic effect in a patient while minimizing undesired side effects. The dosage regimen will be determined by skilled clinicians, based on factors such as the exact nature of the condition being treated, the severity of the condition, the age and general physical condition of the patient, and so on.

The ophthalmic compositions of the present invention will include one or more anti-IL-10 agents, anti-IL-10-R agent, or isolated macrophages and a pharmaceutically acceptable vehicle for said compound(s). Various types of vehicles may be used. The vehicles can be aqueous in nature. The compounds can also be readily incorporated into other types of compositions, such as suspensions, viscous or semi-viscous gels or other types of solid or semi-solid compositions. Suspensions may be preferred for agents which are relatively insoluble in water. The ophthalmic compositions of the present invention may also include various other ingredients, such as buffers, preservatives, co-solvents and viscosity building agents.

The anti-IL-10 agents, anti-IL-10-R agents, or isolated macrophages may be contained in various types of pharmaceutical compositions, in accordance with formulation techniques known to those skilled in the art. For example, the agents may be included in solutions, suspensions and other dosage forms adapted for topical application to the involved tissues, such as tissue irrigating solutions, or injection to the involved tissues. An appropriate buffer system (e.g., sodium phosphate, sodium acetate or sodium borate) may be added to prevent pH drift under storage conditions.

Ophthalmic products are typically packaged in multidose form. Preservatives are thus generally required to prevent microbial contamination during use. Examples of suitable preservatives include: benzalkonium chloride, thimerosal, chlorobutanol, methyl paraben, propyl paraben, phenylethyl alcohol, edetate disodium, sorbic acid, polyquaternium-1, or other agents known to those skilled in the art. Such preservatives are typically employed at a level of from about 0.001 to about 1.0 percent by weight, based on the total weight of the composition (wt. %).

Some of the anti-IL-10 agents and/or anti-IL-10-R agents may have limited solubility in water and therefore may require a surfactant or other appropriate co-solvent in the composition. Such co-solvents include: polyethoxylated castor oils, Polysorbate 20, 60 and 80; Pluronic Registered TM F-68, F-84 and P-103 (BASF Corp., Parsippany N.J., USA); cyclodextrin; or other agents known to those skilled in the art. Such co-solvents are typically employed at a level of from about 0.01 to about 2 wt. %.

Physiologically balanced irrigating solutions can be used as pharmaceutical vehicles for the anti-IL-10 agent(s) and/or anti-IL-10-R agent(s) when the compositions are administered intraocularly. As used herein, the term “physiologically balanced irrigating solution” means a solution which is adapted to maintain the physical structure and function of tissues during invasive or noninvasive medical procedures. This type of solution will typically contain electrolytes, such as sodium, potassium, calcium, magnesium, and/or chloride; an energy source, such as dextrose; and a buffer to maintain the pH of the solution at or near physiological levels. Various solutions of this type are known (e.g., Lactated Ringers Solution). BSS Registered TM Sterile Irrigating Solution and BSS Plus Registered TM Sterile Intraocular Irrigating Solution (Alcon Laboratories, Inc., Fort Worth, Tex., USA) are examples of physiologically balanced intraocular irrigating solutions.

The specific type of formulation selected will depend on various factors, such as anti-IL-10 agent(s) and/or anti-IL-10-R agent(s) being used, the dosage frequency, and the location of the neovascularization being treated. Topical ophthalmic aqueous solutions, suspensions, ointments, and gels are the preferred dosage forms for the treatment of neovascularization in the front of the eye (the cornea, iris, trabecular meshwork); or neovascularization of the back of the eye if anti-IL-10 agent(s) and/or anti-IL-10-R agent(s) can be formulated such that it can be delivered topically and the agent is able to penetrate the tissues in the front of the eye. Intravitreous injectionable ophthalmic preparations are generally preferable for treatment of neovascularization of tissues of the back of the eye.

Viscosity greater than that of simple aqueous solutions may be desirable to increase ocular absorption of the active compound, to decrease variability in dispensing the formulations, to decrease physical separation of components of a suspension or emulsion of formulation and/or otherwise to improve the ophthalmic formulation. Such viscosity building agents include, for example, polyvinyl alcohol, polyvinyl pyrrolidone, methyl cellulose, hydroxypropyl methylcellulose, hydroxyethyl cellulose, carboxymethyl cellulose, hydroxypropyl cellulose or other agents known to those skilled in the art. Such agents are typically employed at a level of from about 0.01 to about 2 wt. %.

As indicated above, use of an anti-IL-10 agent(s) and/or anti-IL-10-R agent(s) to prevent or reduce angiogenesis in ophthalmic tissues represents several aspects of the invention. The anti-IL-10 agent(s) and/or anti-IL-10-R agent(s) may also be used as an adjunct to ophthalmic surgery, such as by vitreal or subconjunctival injection following ophthalmic surgery. The anti-IL-10 agent(s) and/or anti-IL-10-R agent(s) may be used for acute treatment of temporary conditions, or may be administered chronically, especially in the case of degenerative disease. The compounds may also be used prophylactically, especially prior to ocular surgery or noninvasive ophthalmic procedures, or other types of surgery.

Endogenous Therapy

The principles of gene therapy for the production of therapeutic products, herein for example IL-10 antisense nucleic acids and IL-10 siRNAs, within the body include the use of delivery vehicles (termed vectors) that can be non-pathogenic viral variants, lipid vesicles (liposomes), carbohydrate and/or other chemical conjugates of nucleotide sequences encoding the therapeutic protein or substance. These vectors are introduced into the body's cells by physical (direct injection), chemical, or cellular receptor mediated uptake. Once within the cells, the nucleotide sequences can be made to produce the therapeutic substance within the cellular (episomal) or nuclear (nucleus) environments. Episomes usually produce the desired product for limited periods whereas nuclear incorporated nucleotide sequences can produce the therapeutic product for extended periods including permanently.

In clinical settings, the gene delivery systems for therapeutic anti IL-10 agents or anti-IL-10-R agents can be introduced into a patient (or non-human animal) by any of a number of methods, each of which is known in the art. For example, a pharmaceutical preparation of the gene delivery system can be introduced systemically, e.g. by intravitreous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof.

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, and the transfected cells are expanded in number and then reimplanted in the patient. In in vitro gene transfer, the transformed cells are cells growing in culture, such as tissue culture cells, and not particular cells from a particular patient. These “laboratory cells” are transfected, and the transfected cells are selected and expanded for either implantation into a patient or for other uses. In vivo gene transfer involves introducing the DNA into the cells of the patient when the cells are within the patient. In vivo gene transfer also involves introducing the DNA specifically into the ocular endothelial cells of the patient using gene therapy vectors containing endothelial specific promoters. All three of the broad-based categories described above may be used to achieve gene transfer in vivo, ex vivo, and in vitro.

Mechanical (i.e., physical) methods of DNA delivery can be achieved by microinjection of DNA into germ or somatic cells, pneumatically delivered DNA-coated particles such as the gold particles used in a “gene gun,” and inorganic chemical approaches such as calcium phosphate transfection. The plasmid DNA may or may not integrate into the genome of cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Particle-mediated gene transfer may also be employed for injecting DNA into cells, tissues, and organs. With a particle bombardment device, or “gene gun,” a motive force is generated to accelerate DNA-coated high density particles (such as gold or tungsten) to a high velocity that allows penetration of the target organs, tissues, or cells. Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. The techniques of particle-mediated gene transfer and electroporation are well known to those of ordinary skill in the art.

Chemical methods of gene therapy involve carrier-mediated gene transfer through the use of fusogenic lipid vesicles such as liposomes or other vesicles for membrane fusion. A carrier harboring a DNA or protein of interest can be conveniently introduced into body fluids or the bloodstream and then site specifically directed to the target organ or tissue in the body. Cell or organ-specific DNA-carrying liposomes, for example, can be developed and the foreign DNA carried by the liposome absorbed by those specific cells. Injection of immunoliposomes that are targeted to a specific receptor on certain cells can be used as a convenient method of inserting the DNA into the cells bearing that receptor. Another carrier system that has been used is the asialoglycoprotein/polylysine conjugate system for carrying DNA to hepatocytes for in vivo gene transfer.

Transfected DNA may also be complexed with other kinds of carriers so that the DNA is carried to the recipient cell and then deposited in the cytoplasm or in the nucleoplasm. DNA can be coupled to carrier nuclear proteins in specifically engineered vesicle complexes and carried directly into the nucleus.

Carrier mediated gene transfer may also involve the use of lipid-based compounds which are not liposomes. For example, lipofectins and cytofectins are lipid-based positive ions that bind to negatively charged DNA and form a complex that can ferry the DNA across a cell membrane. Another method of carrier mediated gene transfer involves receptor-based endocytosis. In this method, a ligand (specific to a cell surface receptor) is made to form a complex with a gene of interest and then injected into the bloodstream. Target cells that have the cell surface receptor will specifically bind the ligand and transport the ligand-DNA complex into the cell.

Biological gene therapy methodologies employ viral vectors to insert genes into cells. Viral vectors that have been used for gene therapy protocols include, but are not limited to, retroviruses, other RNA viruses such as poliovirus or Sindbis virus, adenovirus, adeno-associated virus, herpes viruses, SV 40, vaccinia, lentivirus, and other DNA viruses. Replication-defective murine retroviral vectors are the most widely utilized gene transfer vectors. Murine leukemia retroviruses are composed of a single strand RNA completed with a nuclear core protein and polymerase (pol) enzymes encased by a protein core (gag) and surrounded by a glycoprotein envelope (env) that determines host range. The genomic structure of retroviruses includes gag, pol, and env genes enclosed at the 5′ and 3′ long terminal repeats (LTRs). Retroviral vector systems exploit the fact that a minimal vector containing the 5′ and 3′ LTRs and the packaging signal are sufficient to allow vector packaging and infection and integration into target cells providing that the viral structural proteins are supplied in trans in the packaging cell line.

Fundamental advantages of retroviral vectors for gene transfer include efficient infection and gene expression in most cell types, precise single copy vector integration into target cell chromosomal DNA and ease of manipulation of the retroviral genome. For example, altered retrovirus vectors have been used in ex vivo methods to introduce genes into peripheral and tumor-infiltrating lymphocytes, hepatocytes, epidermal cells, myocytes or other somatic cells (which may then be introduced into the patient to provide the gene product from the inserted DNA).

The adenovirus is composed of linear, double stranded DNA complexed with core proteins and surrounded with capsid proteins. Advances in molecular virology have led to the ability to exploit the biology of these organisms to create vectors capable of transducing novel genetic sequences into target cells in vivo. Adenoviral-based vectors will express gene product peptides at high levels. Adenoviral vectors have high efficiencies of infectivity, even with low titers of virus. Additionally, the virus is fully infective as a cell-free virion so injection of producer cell lines is not necessary. Another potential advantage to adenoviral vectors is the ability to achieve long term expression of heterologous genes in vivo.

Gene therapy also contemplates the production of a protein or polypeptide where the cell has been transformed with a genetic sequence that turns off the naturally occurring gene encoding the protein, i.e., endogenous gene-activation techniques.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

Volume of the neovascular complex was examined in IL-10 knockout mice. Knockout mice were purchased commercially (NCI, Frederick, Md.). All work was carried out per the ARVO guidelines for animal use in research. Rupture of Bruch's membrane with laser was used to initiate chorioretinal neovascularization in 5-7 week old mice as described in Nambu et al. ((2003) Invest Ophthalmol Vis Sci 44: 3650). Seven days after laser, the mice were anesthetized and perfused intraventricularly with fluorescein-labeled dextran. The mice were euthanized with CO₂ inhalation and the eyes harvested for tissue processing. A dissecting microscope was used to remove the cornea and lens and gently separate the retina from the underlying choroid and sclera. Microscissors were used to make four radial incisions in the sclerochoroidal ‘eyecup’ in order to prepare choroidal flat mounts on glass slides. The tissues were incubated in 4% paraformaldehyde for 45 minutes and washed three times with 3% bovine serum albumin. The tissues were then counter-stained with Cy-3 conjugated anti-mouse elastin antibody for 1 hour and washed three times with 3% BSA. The choroidal flat mounts were analyzed for presence of CNV by confocal microscopy. Extent of choroidal neovascularization was quantified by Metamorph™ imaging software.

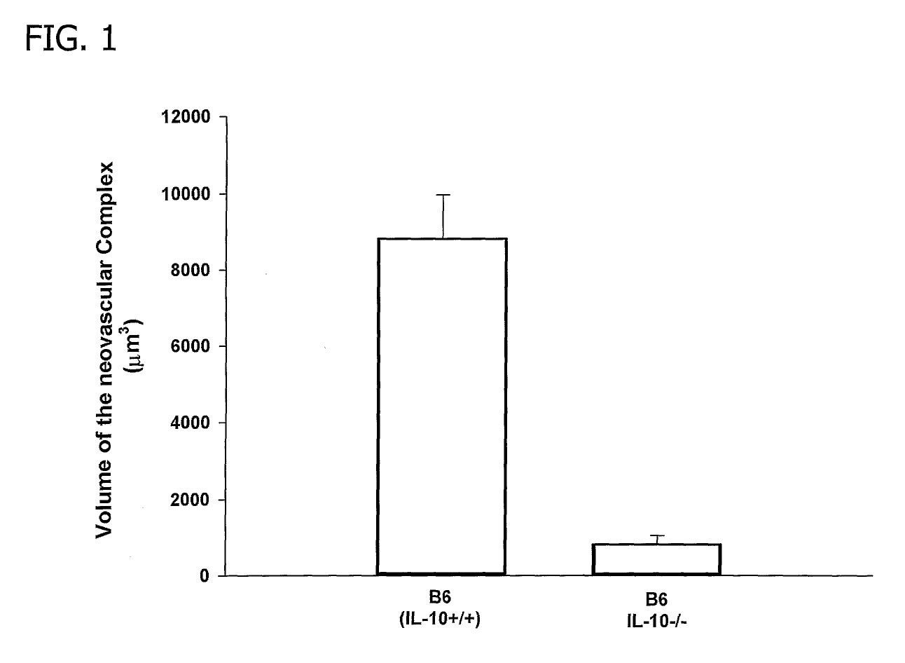

Results showed that choroidal neovascularization was significantly reduced in the IL-10 knockout mice (IL-10−/−) as compared to wild type mice (IL-10+/+) (see e.g. FIG. 1).

### Example 2

Volume of the neovascular complex was examined in wild type mice treated with neutralizing IL-10 antibody (Genzyme, Inc., Cambridge, Mass.). Laser treatment and harvesting of the mouse eyes were as described above. Mice received intravenous injections of antibodies in order to neutralize the IL-10 cytokine one day prior to laser photocoagulation (day −1). Antibody injections were repeated on days 0, 1, 3, and 5 prior to harvesting the eyes on day 7 for analysis of choroidal neovascularization. Control mice receive the same dosages of an isotype-matched antibody (Genezyme, Inc., Cambridge, Mass.).

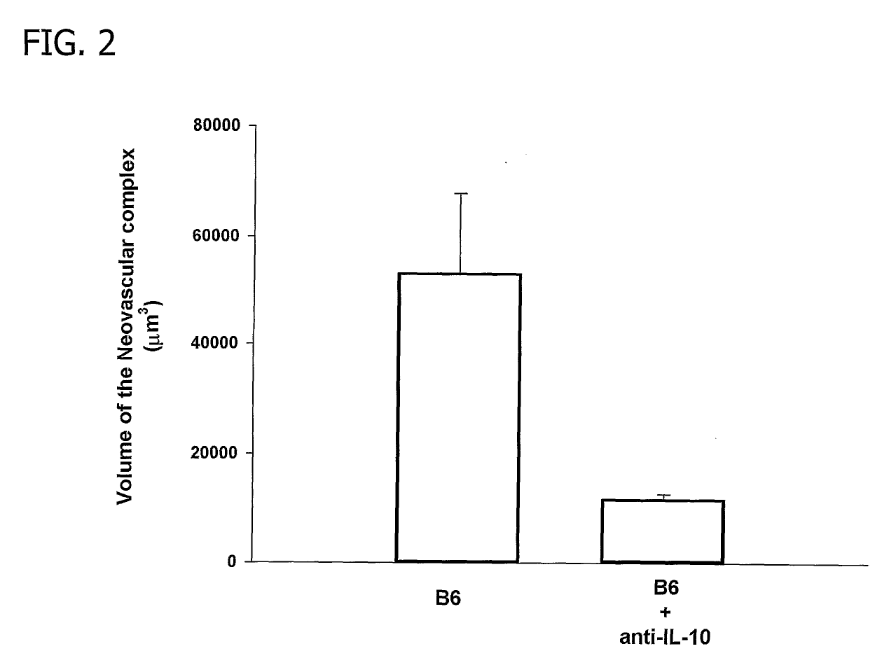

Results showed that choroidal neovascularization was significantly reduced in the mice treated with neutralizing IL-10 antibody (see e.g. FIG. 2).

### Example 3

Bone marrow chimeras were produced as described in Bonfoco et al. (1998) Immunity 9: 711. Host mice receive 1100 R of external beam radiation in order to deplete bone marrow cells. Syngeneic donor mice are euthanized and bone marrow cells are harvested from the proximal limb bones. Host mice receive 1×10⁸ bone marrow cells intravenously. The mice were observed for three weeks in order to allow the donor cells to replenish the host marrow and form true chimeras prior to assessing the choroidal neovascularization response. Bone marrow cells in the host animal were derived from and function like donor marrow cells. Bone marrow chimera experiments were performed with meticulous age-matched chimera controls for all the cytokine knockout mice tested.

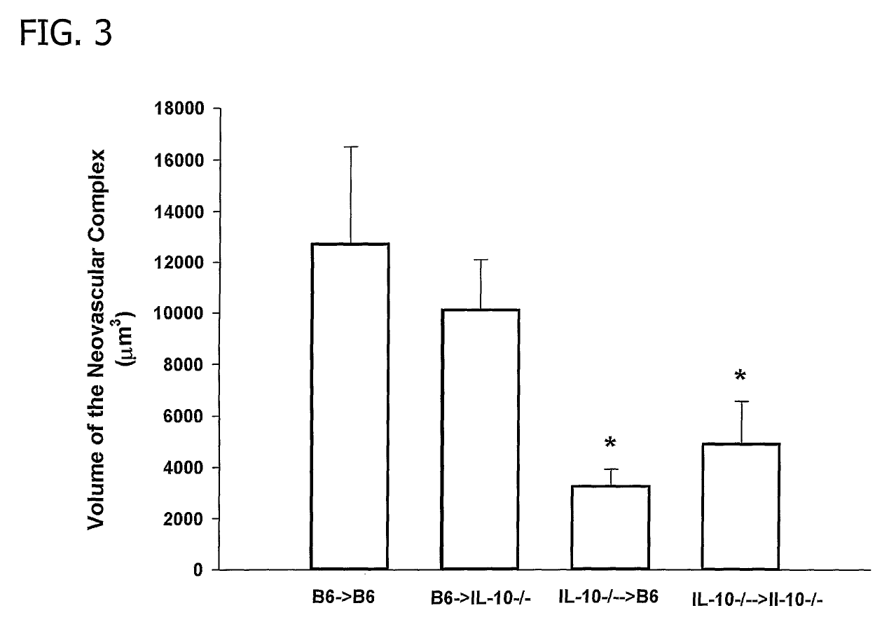

Results showed that inhibition of choroidal neovascularization in IL-10 knock-out mice was reversed after bone marrow of the knock-out mice was replaced with wild type (i.e., IL-10 producing) bone marrow (see e.g. FIG. 3). Furthermore, bone marrow not having the capacity of IL-10 formation successfully inhibited the ability of wild type mice to form choroidal neovascularization after generation of bone marrow chimeras (see e.g. FIG. 3).

### Example 4

Exogenous IL-10 was injected into the eyes of IL-10 knockout mice. Laser treatment and harvesting of the mouse eyes were as described above.

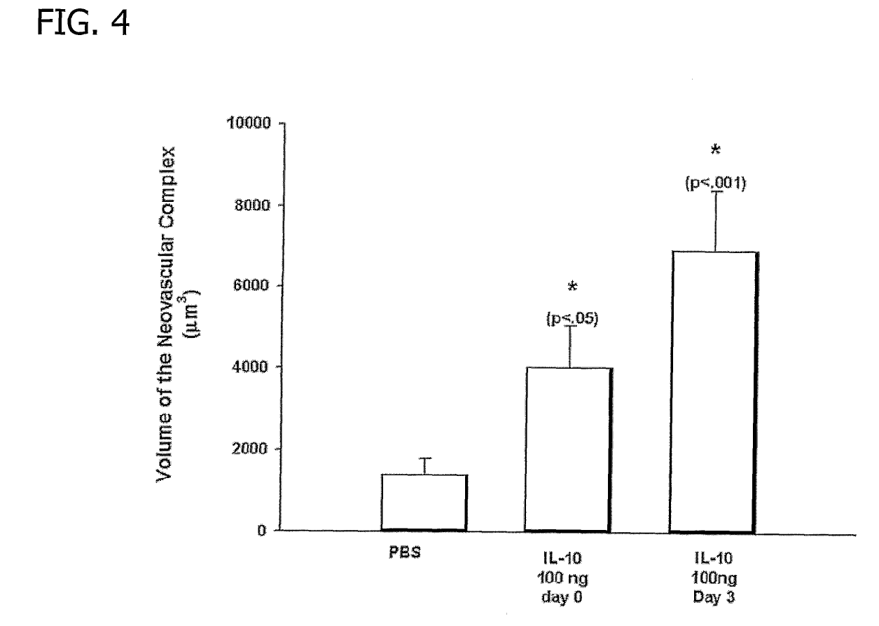

Results showed that IL-10 administered intravitreally on the day of laser treatment or three days after laser reversed the inhibition choroidal neovascularization (see e.g. FIG. 4).

### Example 5

Macrophage migration into the neovascular complex was analyzed in response to the presence (wild type mice) or absence of IL-10 (IL-10 knock out mice). Sclerochoroidal flat mounts were prepared 7 days after laser treatment from wild type or IL10 knock out mouse eyes. PE-conjugated anti-CD11b antibody (1:100) or isotype-matched control antibody (BD Biosciences, San Jose, Calif.) were used to stain the mounts for 1 hour at room temperature and then washed with PBS and analyzed by 3-D confocal microscopy. Numbers of macrophages (CD11b+) were counted per lesion and average numbers were represented. The lesions were also stained in flat mounts for neutrophils with PE-conjugated Gr-1 antibody, dendritic cells with PE-conjugated anti-CD11c, and T cells with PE-conjugated CD3 antibody (BD Biosciences, San Diego, Calif.).

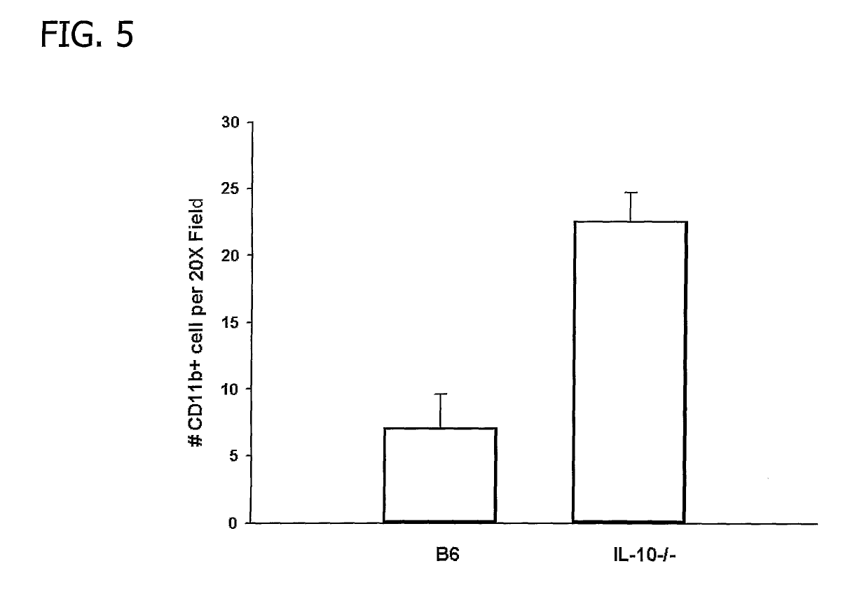

Results showed that IL-10 knock out mice had substantially more macrophages as compared to wild type mice, thus demonstrating that IL-10 inhibits migration of macrophages into the neovascular complex (see e.g. FIG. 5).

### Example 6

Macrophage migration into the neovascular complex was analyzed in response to the presence (wild type mice) or absence of IL-10 (neutralizing IL-10 antibodies injected into wild type mice). Sclerochoroidal flat mounts were prepared as above. Injection of antibody was as described above.

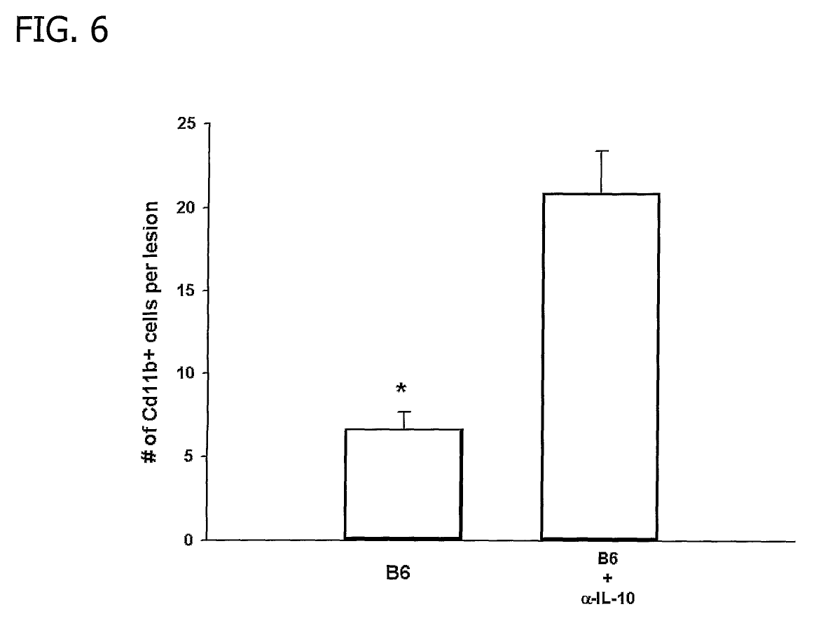

Results showed that wild type mice treated with neutralizing IL-10 antibody had substantially more macrophages as compared to wild type mice, thus demonstrating that IL-10 inhibits migration of macrophages into the neovascular complex (see e.g. FIG. 6).

### Example 7

Macrophages were used to proactively treat the formation of blood vessels in the eye. Native CD11b+ macrophages were purified from mice spleen using the PE-positive selection (SpinSep™, Stem Cell Technologies, Inc.). Various doses of macrophages were then injected into the vitreous cavity of eyes of mice on the same day as the laser treatment described above. Control mice were injected with PBS or other purified immune cells such as CD3+ T cells or CD11c+ dendritic cells using the same purification protocol. Choroidal neovascularization was analyzed on day 7.

Alternatively, native CD11b+ macrophages were purified from GM-CSF cultured macrophages using the PE-positive selection (SpinSep™, Stem Cell Technologies, Inc., Vancouver, BC, Canada). Bone marrow was isolated from proximal limb bones as described previously Inaba, et al. (1992) J. Exp. Med., 176(6): 1693-1702. Briefly, all muscle tissue was removed from the bones, and the bones were washed in 70% alcohol for 5 seconds prior to two washes in PBS. The ends of the bones were cut with scissors and the marrow harvested using a syringe and 25-gauge needle to flush the bones with complete RPMI. 2×10⁶ cells in 10 ml complete RPMI and 1000 U/ml GM-CSF were cultured for 10 days in 100 mm petri dishes. On day 3 and 6, an additional 500 U/ml and 1000 U/ml GM-CSF were added respectively. On day 10, the non-adherent cells containing dendritic cells were discarded. The adherent cells were mechanically removed and harvested with a cell scraper. CD11b+ cells were then isolated by positive selection.

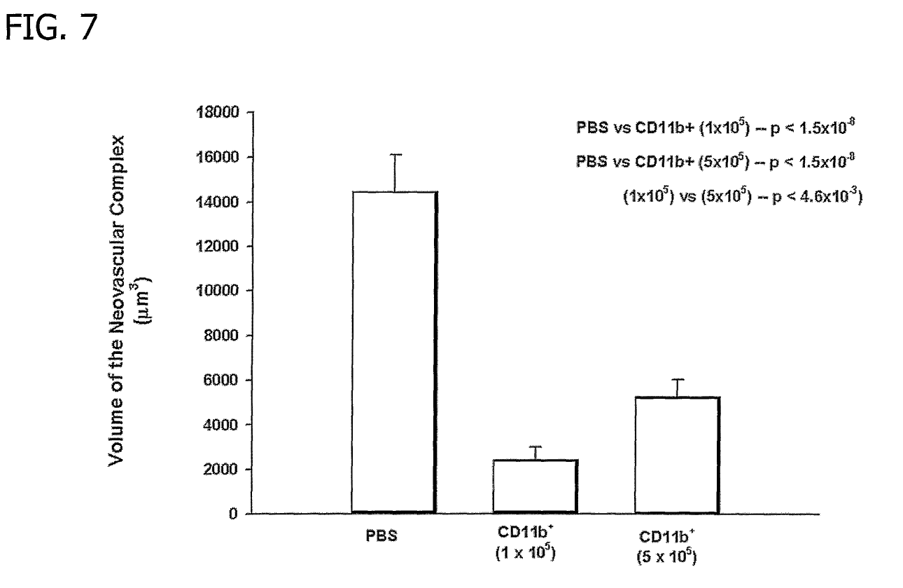

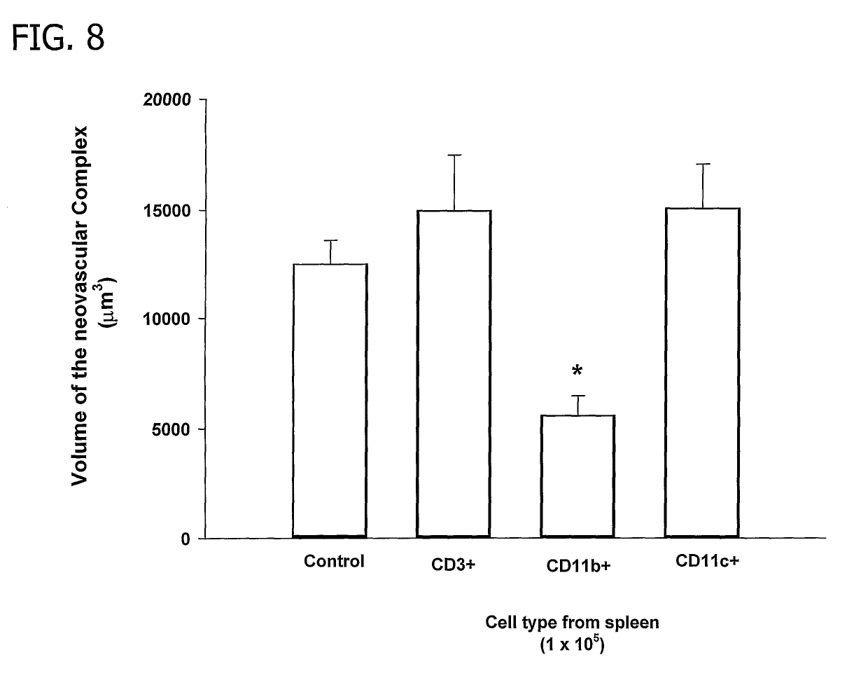

Results showed that macrophages injected into the eye were able to successfully inhibit choroidal neovascularization (see e.g. FIG. 7) while T lymphocytes (CD3+) and dendritic cells (CD11c+) fail to do so (see e.g. FIG. 8).

### Example 8

The following experiment suggests macrophages signal through the Fas-FasLigand death pathway and inhibit nascent vascular endothelial cell growth in the eye. Fas ligand expressed on macrophages may signal through Fas expressed on vascular endothelial cells and induce vascular endothelial cell apoptosis. Macrophage isolation and injection was as described above. Macrophages were obtained from animals deficient in Fas (Ipr-) or FasLigand (gld-).

CFSE [1 μL of 5 mM CFSE] was added to 1×10⁶ purified macrophages (1:1000 dilution) from either C57BL6-wt or gld mice. Cells were incubated 10 minutes at 37° C. in a water bath and washed three times. Labeling was confirmed by fluorescent microscopy

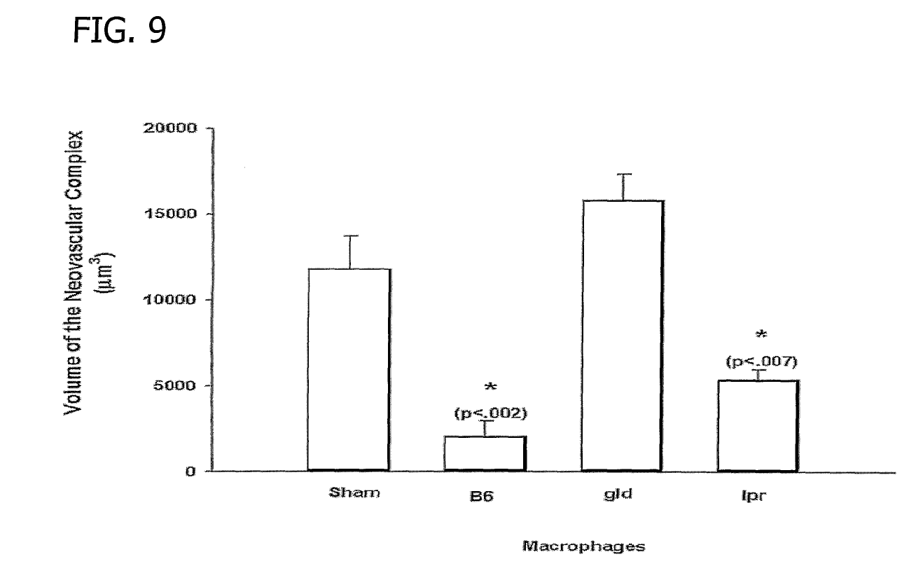

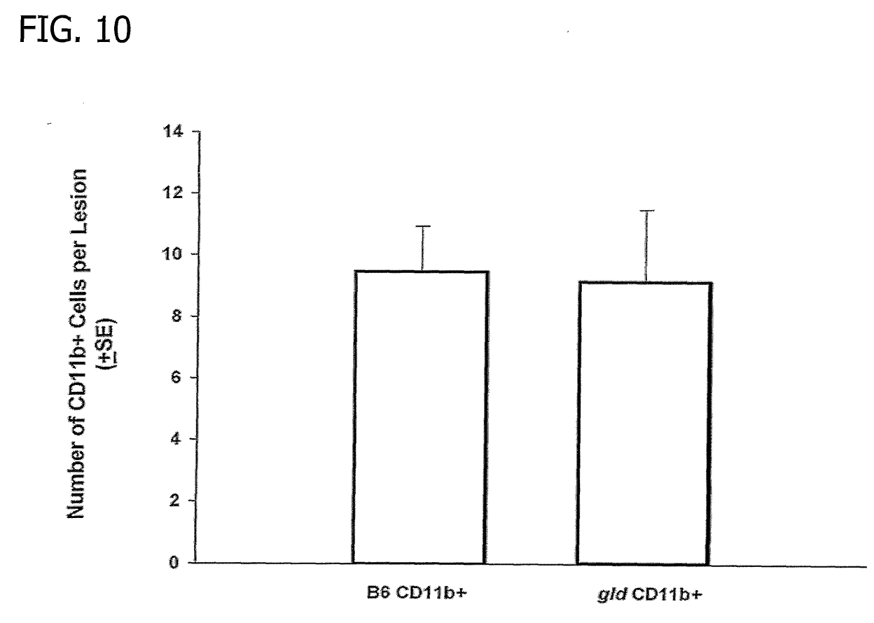

Results showed that Cd11b+ macrophages derived from wild type and Fas-deficient (Ipr-derived) mice inhibit choroidal neovascularization while CD11b+ macrophages lacking Fas Ligand (gld-derived) fail to inhibit choroidal neovascularization (See e.g. FIG. 9). This effect is not due to the inability of gld macrophages to home to the laser lesion after injection since there was no difference in the number of CFSE-labeled Wt or gld macrophages per lesion when they were examined 3 days after injection (see, e.g., FIG. 10). This suggests that signaling by macrophages through the Fas Ligand pathway inhibits vascular growth and choroidal neovascularization.

### Example 9

The effect of the inhibition of macrophage recruitment on choroidal neovascularization was examined by systemic injection of anti-CD11b or anti-F4/80, treatments known to prevent entry of macrophages into tissue (Gordon, et al. (1995) J Neuroimmunol, 62(2): 153-60).

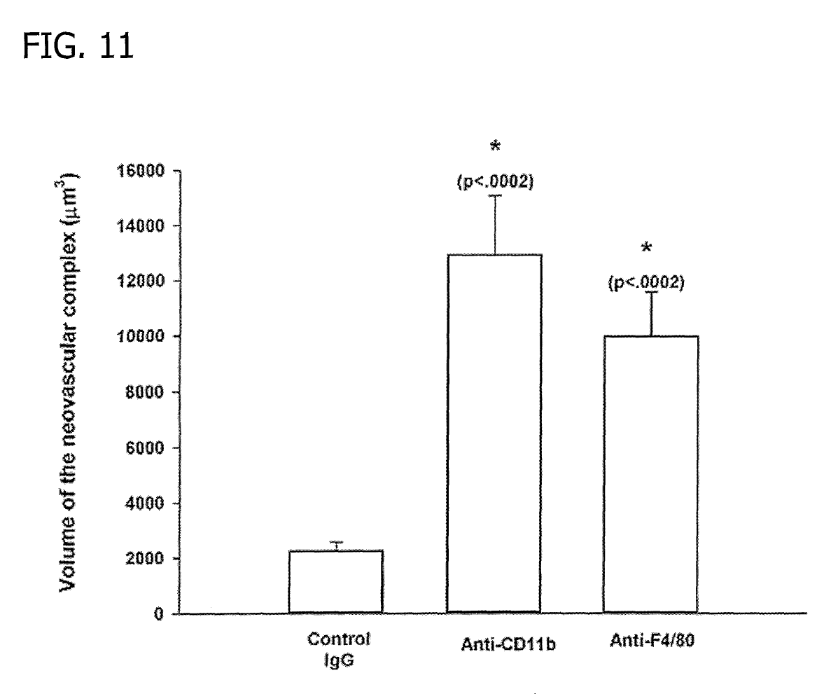

Results showed that depletion of CD11b+ cells with anti-CD11b as well as depletion of macrophages with anti-F4/80 led to significantly increased neovascularization (see, e.g., FIG. 11). Examination of the lesions in treated mice revealed that both treatments abolished the migration of macrophages into the laser lesions (not shown). This further suggests that the presence of macrophages is inhibitory to angiogenesis in the retina.

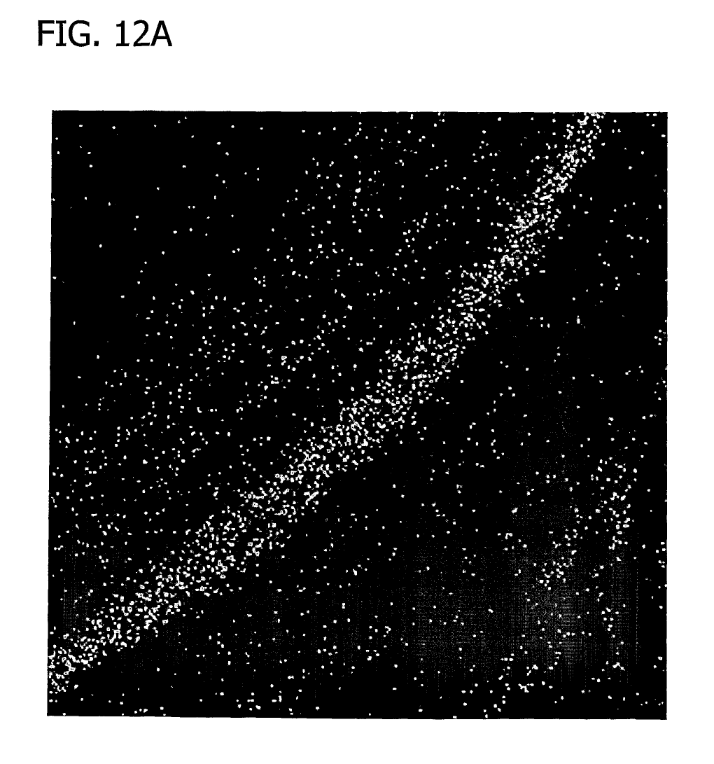

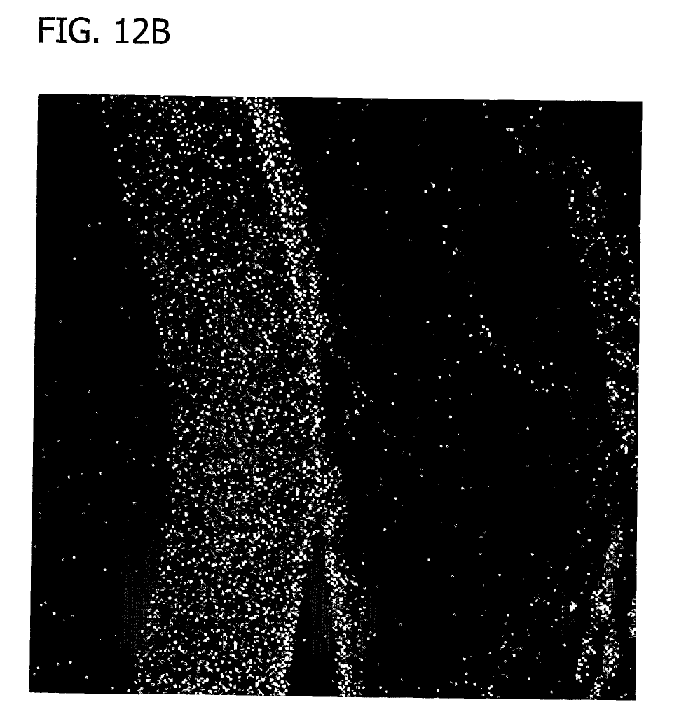

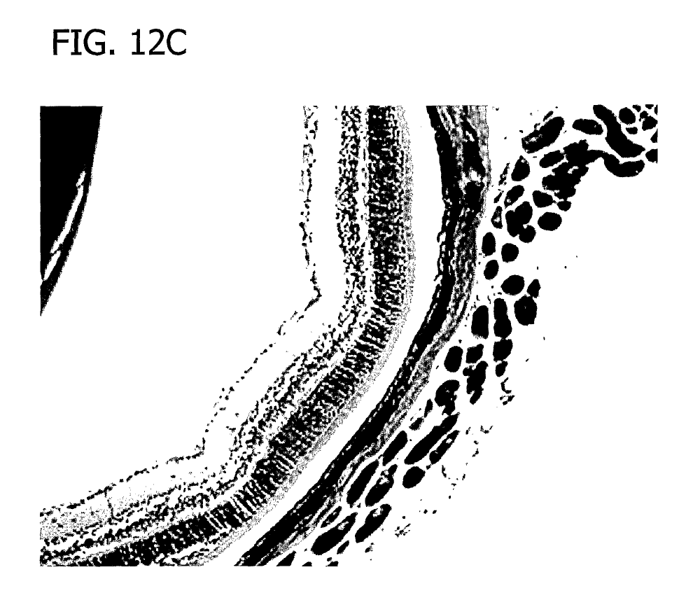

The ability of high levels of IL-10 to prevent macrophage entry into the eye resulting in increased pathologic choroidal neovascularization was examined in transgenic (Tg) mice. A transgenic mouse overexpressing IL-10 in the retinal pigment epithelium (RPE) using the human VMD2 promoter was developed. These IL-10 transgenic mice (or VMD2-IL-10) express high levels of secreted IL-10 in the retina (see e.g., FIG. 12B) compared to transgene negative mice (see e.g., FIG. 12A), but have completely normal retinal architecture (see e.g., FIG. 12C).

### Example 10

VMD2-IL-10 transgenic (Tg) mice were constructed to overexpress IL-10 or FN-14 in RPE cells. VMD2 (Bestrophin) localizes to the basolateral plasma membrane of the RPE (Marmorstein et al. (2000) PNAS USA, 97(23): 12758-63). The pVMD2-placF was provided by Dr. Noriko Ezumi, (Johns Hopkins Medical School). We removed the VMD2 promoter and cloned it into the pCI plasmid (Promega, Madison, Wis.) replacing the CMV promoter and then placed the IL-10 ORF downstream. The VMD2-FN-14 was made by replacing the IL-10 ORF with the FN-14 ORF (Wiley et al. (2001) Immunity, 15(5): 837-46). Transgenic mice were produced by injecting fertilized mouse oocytes with transgene DNA by standard protocols in the Department of Ophthalmology and Visual Science Molecular Biology Core facility. Founders were screened by PCR and used for breeding. IL-10 expression was verified by RT-PCR and immunohistochemistry. Biomicroscopic examination of the anterior and posterior segments of the eye and histopathologic analysis of ocular tissues showed no overt abnormalities. The mice were viable and had normal life spans compared to littermate controls (not shown).

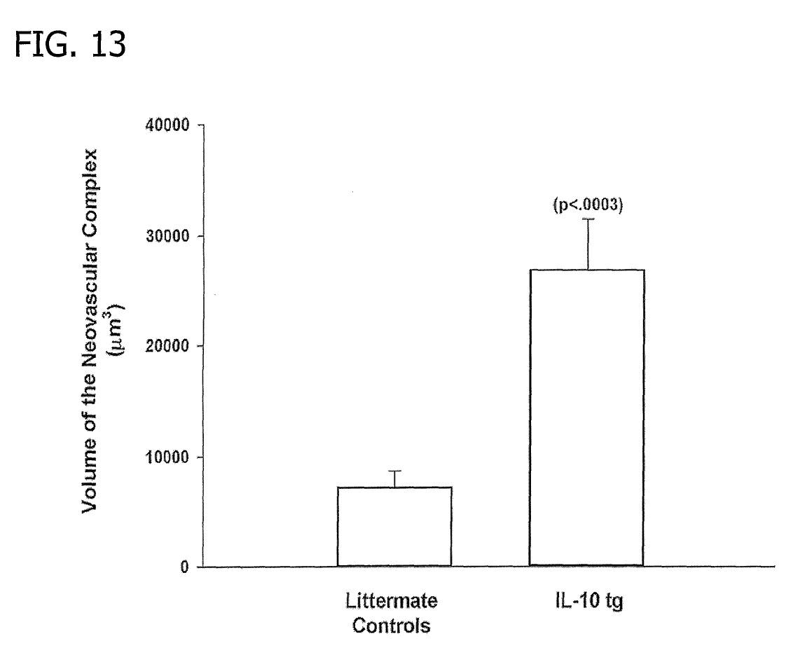

When choroidal neovascularization was induced by laser treatment (FIG. 13), neovascularization was substantially elevated over controls. In addition, VMD2-IL-10 Tg mice did not show macrophage infiltrates into the choroidal neovascularization lesions (not shown). As compared to the IL-10 transgenic mouse, choroidal neovascularization was comparable to controls in VMD2-FN-14 mice. Thus direct injection of IL-10 and transgenic overexpression of IL-10 increased new vessel formation.

### Example 11

Macrophages are known to both promote and inhibit inflammatory responses, but they are not typically FasL+ unless stimulated. The ability of macrophages to activate or at least interact with the damaged retinal tissue to promote CD95L expression leading to acquisition of killing function was examined.

Purified CD11b-F4/80 macrophages were placed into 96 well flat bottom tissue culture plates (1×10⁵ per well). Cells were treated with LPS (0.1 μg/ml) or necrotic retinal cells for three hours at 37°, 5% CO₂ in complete RPMI. L1210-Fas target cells (2×10⁴ cells labeled with ³H-thymidine) were added and the plates incubated for an additional 16-20 hrs. Cells were harvested by filtration through glass fiber filters (Packard Instruments, Meriden, Conn.) using a Filtermate 96 cell harvester (Packard Instruments) and counted on a microplate scintillation counter (Packard Instruments). Data are expressed as % Cell Death calculated by: [100×(c.p.m. from L1210-Fas alone—c.p.m. of L1210-Fas+macrophages) per c.p.m. from L1210-Fas alone.

Necrotic retinal cells were prepared from B6 mice. Eyes were removed from euthanized mice and dissected in RPMI complete media. Anterior segment and lens were removed and discarded. The neurosensory retina was gently peeled from the choroid with fine tip forceps and ground between two glass slides. Cells in each were counted and cell number adjusted to 5×10⁷/μl. The retina was placed in RPMI complete media and subjected to four freeze/thaw cycles using liquid nitrogen. Ten (10 μl) of this material was added per well of CD11b+−F4/80+ macrophages.

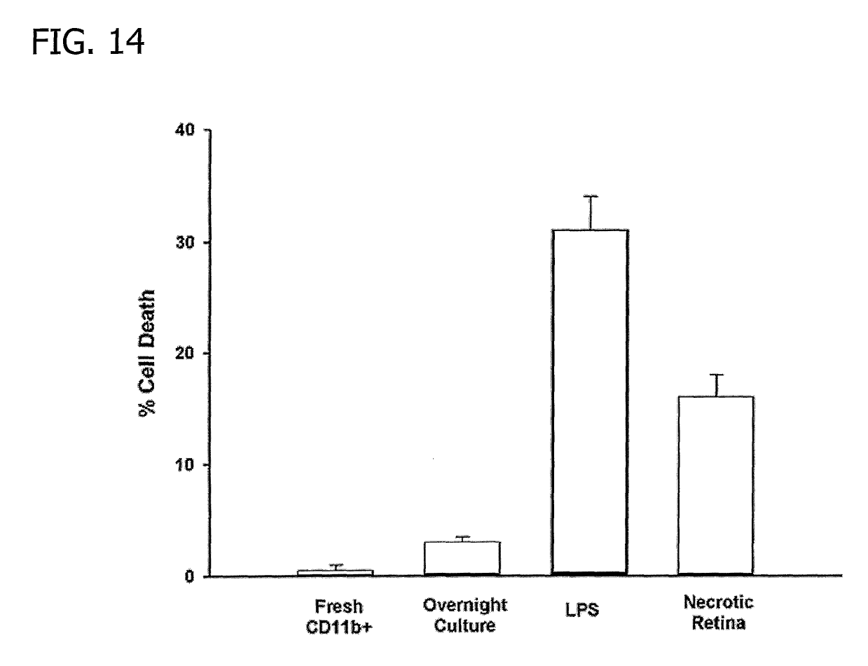

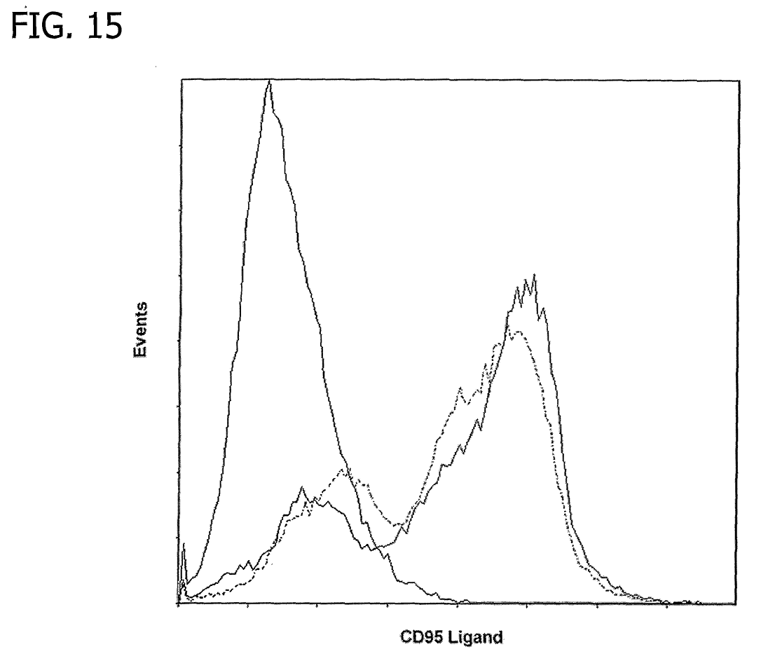

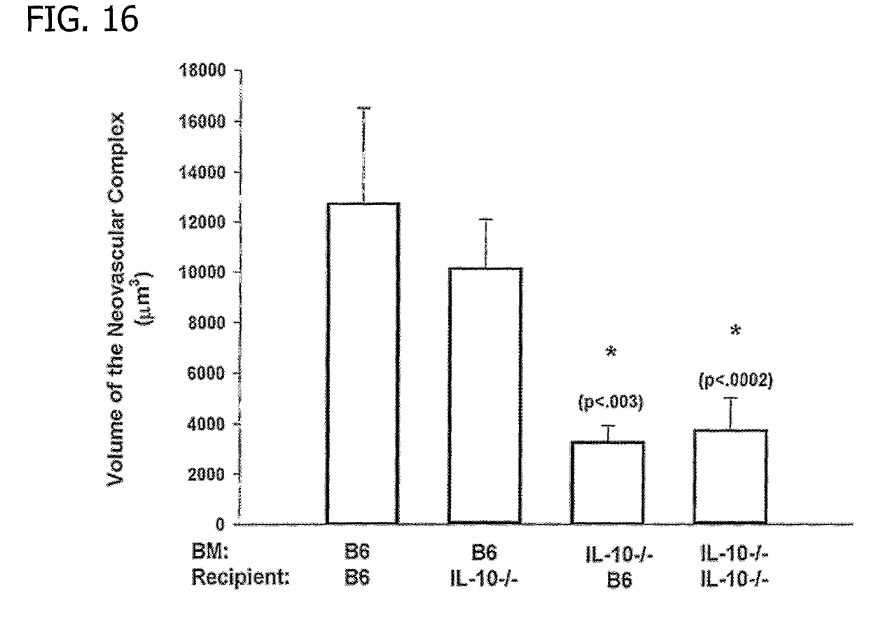

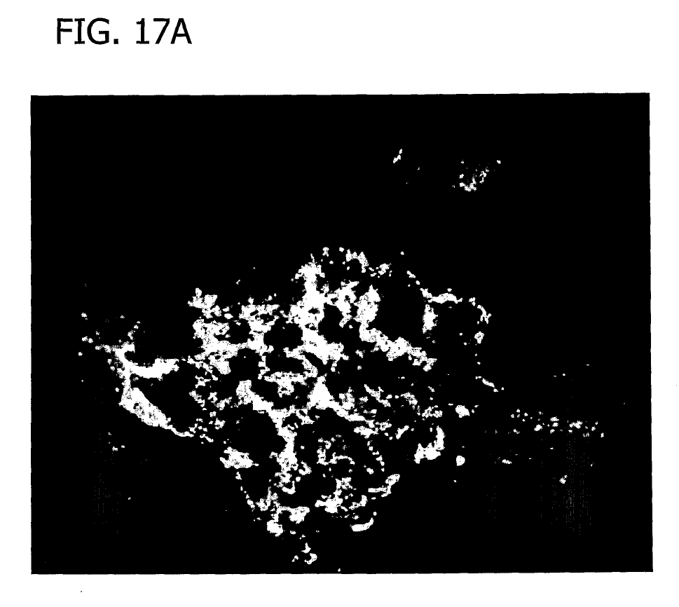

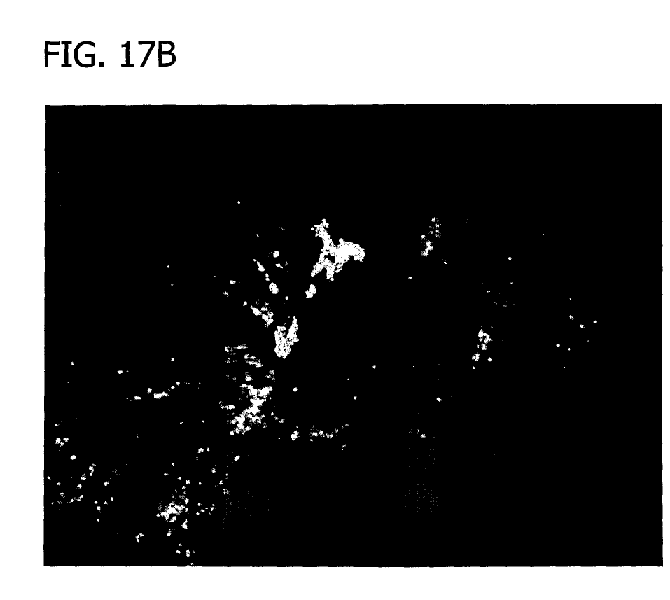

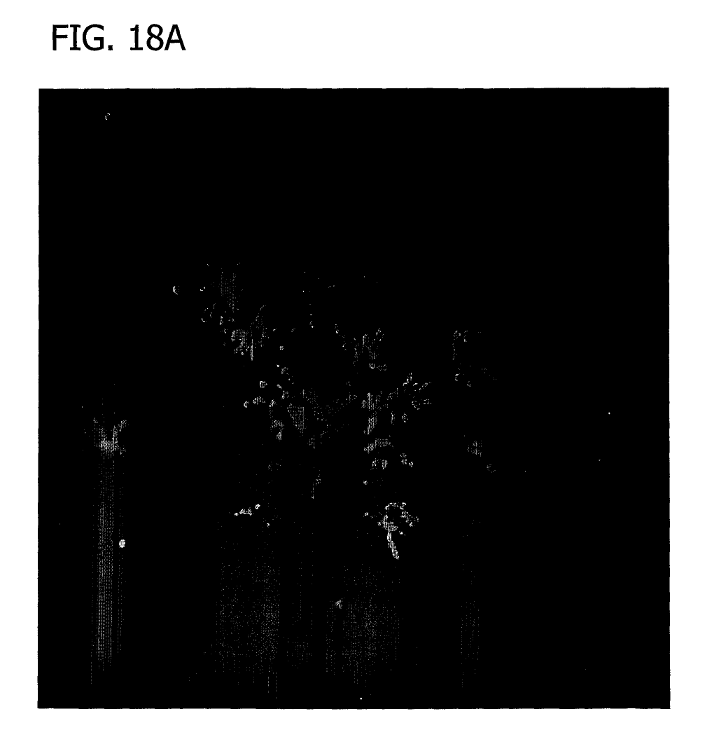

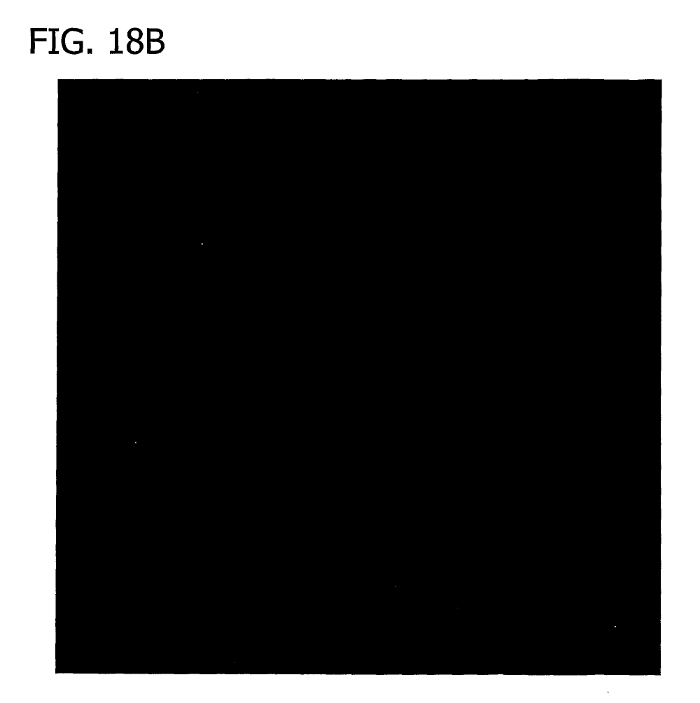

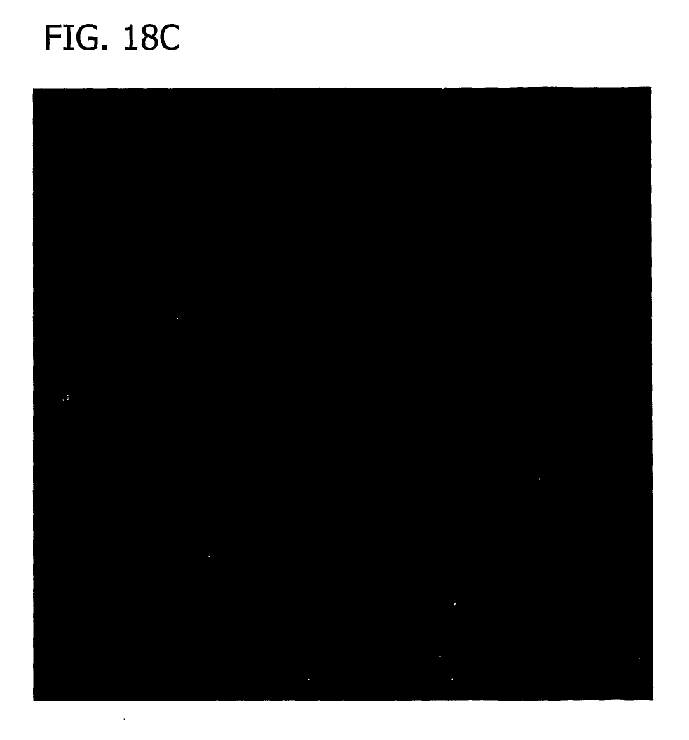

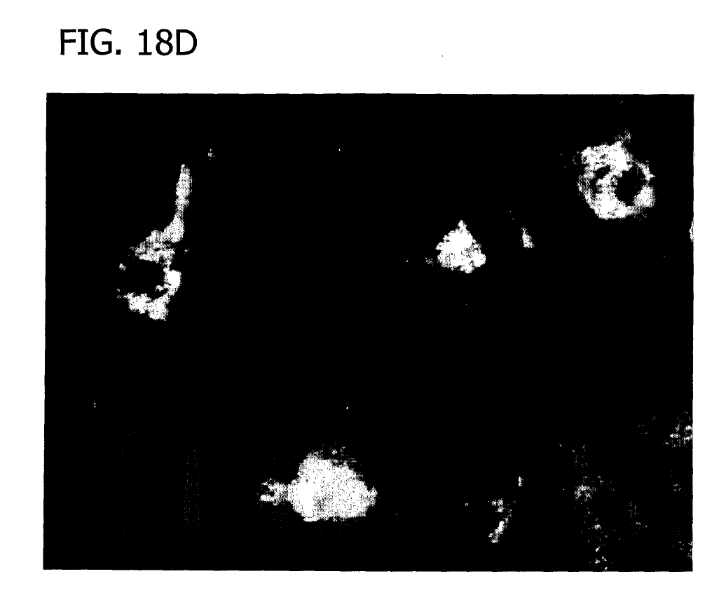

CD11b+ cells purified from spleen were subjected to necrotic cells (retina) or LPS and then tested for their ability to kill CD95+ targets. As shown in FIG. 14, cells fed necrotic cells or treated with LPS were able to kill CD95+ targets. This was likely through upregulation of CD95L on macrophages by both stimuli (see e.g., FIG. 15).

### Example 12

The effect of systemic depletion of phagocytic cells by injection of the compound clodronate encapsulated in liposomes was examined. Liposomes were prepared as described previously in the literature (Espinosa-Heidmann, et al. (2003) Invest Ophthalmol is Sci, 44(8): 3586-92 and Van Rooijen, N. (1989) J Immunol Methods, 124(1): 1-6). Briefly, 75 mg phosphatidylcholine (Sigma-Aldrich, St. Louis, Mo.) and 11 mg Cholesterol (Sigma-Aldrich, St. Louis, Mo.) were dissolved in 20 mL methanol/cholorform (1:1) at 37° C. on a stirrer. The organic phase was removed with vacuum over 2 hours. GFP (BD Biosciences) (100 μg in 100 μL-1 mg/mL) was dissolved in 10 mL PBS and added to the liposomes over a shaker. The mixture was incubated for 2 hours at room temperature and sonicated in a 37° C. water bath for 3 minutes. The liposomes were then incubated at room temperature for 2 hours and centrifuged at 100000×g (45,000 RPM) for 30 minutes at 16° C. The supernatant was removed and the liposomes were resuspended in PBS. GFP labeling of liposomes was confirmed by fluorescent microscopy. Mice were injected with liposomes intravenously on days −2, 0 (day of laser), and day 2. Eyes were harvested for imaging at day 3.

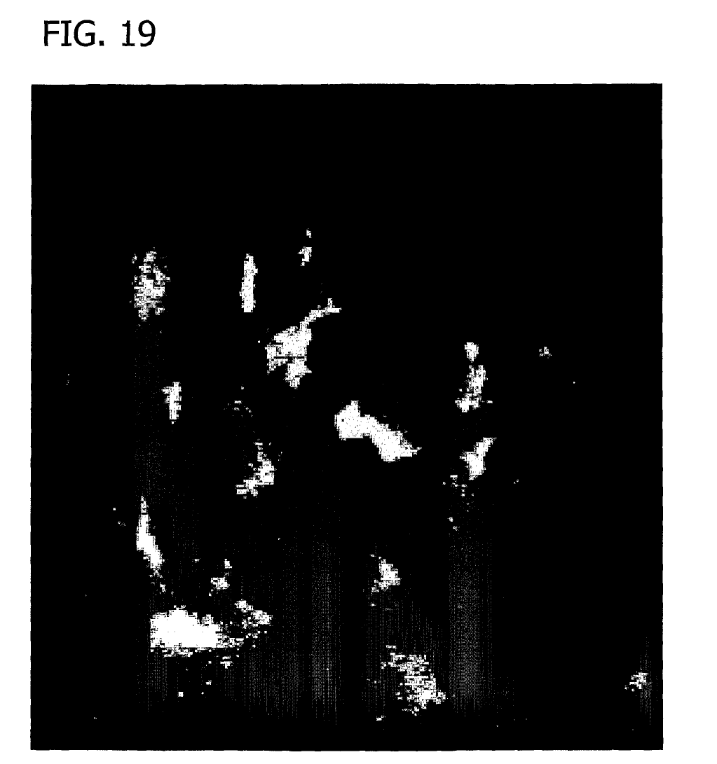

Results showed that liposomes can actually enter developing endothelial cells found in the laser induced neovascular complexes (see, e.g., FIG. 19). This data, coupled with the fact that liposomes are known to enter a wide variety of other cells (Papadimitriou and Antimisiaris (2000) J Drug Target, 8(5): 335-51 and Krasnici, et al. (2003) Int J Cancer, 105(4): 561-7) suggests that the effects observed may be due to the toxicity of the clodronate liposomes directly on sprouting endothelial cells.

1. A method of treating a patient for ophthalmologic neovascularization, the method comprising
administering an anti-interleukin-10 agent, or a pharmaceutical salt thereof, to an eye of a patient in need thereof to decrease the amount of interleukin-10 in the eye.

2. A method of treating a patient for ophthalmologic neovascularization, the method comprising
injecting an anti-interleukin-10 agent, or a pharmaceutical salt thereof, into an eye of a patient in need thereof to inhibit ophthalmologic neovascularization.

3. The method of claim 1 wherein the anti-interleukin-10 agent is an IL-10 antisense nucleic acid that reduces the expression of interleukin-10.

4. The method of claim 3 wherein IL-10 antisense nucleic acid is administered in an amount of (i) about 10 μg/day to about 3 mg/day; (ii) about 30 μg/day to about 300 μg/day; or (iii) about 100 μg/day.

5. The method of claim 1 wherein the anti-interleukin-10 agent is a (i) double stranded RNA (dsRNA) or (ii) small interfering RNA (siRNA) specific for interleukin-10, wherein the dsRNA or siRNA reduces the expression of interleukin-10.

6. The method of claim 5 wherein the siRNA is administered in an amount that results in an intracellular concentration of siRNA at or near the neovascularization complex of (i) about 1 nM to about 100 nM; (ii) about 2 nM to about 50 nM; or (iii) about 2.5 nM to about 10 nM.

7. The method of claim 1 wherein the anti-interleukin-10 agent is an anti-interleukin-10 antibody.

8. The method of claim 7 wherein the anti-interleukin-10 antibody is administered in an amount of (i) about 0.01 mg to about 5.0 mg; (ii) about 0.05 mg to about 2.5 mg; (iii) about 0.1 mg to about 1.0 mg; or (iv) about 0.3 mg to about 0.5 mg.

9. The method of claim 1 wherein the anti-interleukin-10 agent is an interleukin-10 inhibitor.

10. The method of claim 9 wherein the interleukin-10 inhibitor is a polymethoxylated flavone.

11. The method of claim 10 wherein the polymethoxylated flavone is administered in an amount of (i) about 0.1 μg to about 1.0 g or (ii) about 1 mg to about 100 mg.

12. The method of claim 1 wherein the anti-interleukin-10 agent is an interleukin-10 agonist.

13. The method of claim 1 wherein the anti-interleukin-10 agent is administered by injection.

14. The method of claim 1 wherein the anti-interleukin-10 agent is administered by a gene delivery system.

15. The method of claim 1 further comprising the step of administering isolated macrophages to the tissue of the eye of the patient in need thereof to decrease the volume of a neovascularization complex within the treated eye.

16. A method of treating a patient for ophthalmologic neovascularization, the method comprising
administering isolated macrophages to an eye of a patient in need thereof to decrease a volume of a neovascularization complex within the treated eye.

17. The method of claim 16 wherein the isolated macrophages are administered in an amount of (i) about 1×10⁴ to about 1×10⁶ macrophages or (ii) about 1×10⁵ to about 5×10⁵ macrophages.

18. The method of claim 16 wherein the isolated macrophages are administered by injection.
